(19) United States
(12) Patent Application Publication
(10) Pub. No.: US 2006/0116762 A1
(43) Pub. Date: Jun. 1, 2006

(54) Aspheric lenticule for keratophakia

(76) Inventors: Xin Hong, Arlington, TX (US); Xiaoxiao Zhang, Fort Worth, TX (US); Charles Freeman, Granbury, TX (US); Mutlu Karakelle, Forth Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/999,612

(22) Filed: Nov. 30, 2004

(51) Int. Cl. A61F 2/14; A61F 9/008

(52) U.S. Cl. 623/5.16; 623/5.11; 606/5

(57) ABSTRACT

Contour-matching, aspheric lenticules are disclosed for implantation in a subject's cornea to correcting refractive errors. The lenticules include a photoablatable anterior surface and a posterior surface having an aspheric profile that can substantially match the asphericity exhibited by the corneal stromal surface, on which the lenticule is placed. The posterior surface can have a generally concave shape while the anterior surface can have a generally convex shape, though other shapes can also be utilized in some embodiments. In some embodiments, the asphericity of the lenticule's posterior surface can differ from an asphericity exhibited by the corneal stromal surface by less than about 50%, or preferably by less than about 20%.

### TECHNICAL FIELD OF THE INVENTION

The present invention is generally directed to corneal inlay lenses and, more particularly, to photo-ablatable lenticules for implantation in a patient's cornea for correcting a refractive error of the patient's eye.

A procedure commonly known as ablatable-adjustable synthetic keratophakia (ASK) involves incorporating a corneal inlay into a patient's cornea to achieve a desired refractive correction. The inlay can be shaped so as to act as a supplemental lens to correct a refractive error of the patient's eye. The corneal inlays are formed of materials that are biocompatible to the corneal tissue and are ablatable in-situ to modify their shape, and thereby, obtain a desired optical power. Conventional corneal inlay lenses, however, suffer from a number of shortcomings. For example, their surface contours do not provide a good fit with internal corneal surfaces, thereby potentially resulting in corneal damage or vision degradation over time. Additionally, a poorly fit corneal implant can result in a bulging out of the eye's central optical zone and increased spherical aberration.

### BRIEF SUMMARY OF THE INVENTION

Contour-matching, aspheric lenticules are disclosed for implantation in a subject's cornea to correcting refractive errors. The lenticules include a photoablatable anterior surface and a posterior surface having an aspheric profile that can substantially match the asphericity exhibited by the corneal stromal surface, on which the lenticule is placed. The posterior surface can have a generally concave shape while the anterior surface can have a generally convex shape, though other shapes can also be utilized in some embodiments. In some embodiments, the asphericity of the lenticule's posterior surface can differ from an asphericity exhibited by the corneal stromal surface by less than about 50%, or more preferably by less than about 20%.

In another aspect, the aspheric lenticules of the invention can improve image contrast by exhibiting a modulation transfer function in air greater than about 0.2 at a spatial frequency of about one-half (50%) of a cut-off spatial frequency associated with the lenticule (i.e., a spatial frequency at which the modulation transfer function has vanishing values) for a wavelength of about 550 nm and an aperture of about 5 mm. For example, a lenticule having an optical power of about 6 Diopters can exhibit a modulation transfer function greater than 0.2 at a spatial frequency of 30 line pair per millimeter (lp/mm). The lenticule can also be characterized by a modulation transfer function (MTF), calculated in a model eye in which the lenticule is implanted, that is greater than about 0.2 at a spatial frequency of about 100 lp/mm for a wavelength of about 550 nm and pupil size of about 5 mm.

In a related aspect, the aspheric profile of the lenticule's posterior surface can be characterized by the following relation:

$$z = \frac{{cr}^{2}}{1 + \sqrt{1 - ( 1 + k ) {c}^{2} {r}^{2}}} ,$$

wherein

z denotes a sag of the surface parallel to an axis (z) perpendicular to the surface,

c denotes a curvature at a vertex of the profile,

k denotes a conic coefficient, and

r denotes a radial position on the surface.

The curvature constant (c) can be determined based on the desired power of the lenticule, the material from which the lenticule is formed, and the curvature of the other surface of the lenticule in a manner known in the art. In many embodiments, the lenticule can have an optical power in air in a range of about −15 Diopters to about +10 Diopters. Further, the conic constant (k) can be selected to be in a range of about −0.5 to about +0.2, e.g., −0.25.

The anterior surface of the lenticule can also be aspheric so as to minimize spherical aberrations of the lenticule. In some embodiments, the asphericity of the anterior surface can be characterized by the above relation with a conic constant selected so as to minimize, and preferably eliminate, spherical aberrations of the lenticule.

In a further aspect, the invention provides an intracorneal implant that includes an optic having a posterior surface and an anterior surface, where the posterior surface is adapted for placement against an stromal surface of the cornea and has an aspherical profile that substantially conforms with a contour of the stromal surface. The anterior surface is photo-ablatable so as to allow adjusting a refractive correction provided by the optic. The optic can be formed, for example, of silicone, ploymethylmethacrylate, polyvinylpyrrolidine, optical homopolymers and copolymers or other suitable polymeric materials.

In some embodiments, the posterior surface has an aspherical concave profile with an asphericity that is substantially similar to an average asphericity exhibited by convex stromal surfaces of the eyes of a selected group of patients so as to facilitate positioning of the posterior surface against the stromal surface.

In another aspect, the present invention provides a method of correcting a refractive error of a subject's eye that includes cutting a substantially uniform flap in the subject's corneal tissue to expose an internal stromal surface of the cornea, and providing a photoablatable lenticule having a posterior surface exhibiting an aspheric curvature substantially matching an asphericity exhibited by the exposed stromal surface. The lenticule is placed on the exposed stromal surface such that the aspheric surface of the lenticule is in contact with the exposed surface followed by photoablating the lenticule to a selected shape (e.g., by eximer laser ablation) so as to provide a desired refraction correction. The flap is then repositioned on the lenticule.

Further understanding of the invention can be obtained by reference to the following detailed description in conjunction with the associated figures, described briefly below.

### BRIEF DESCRIPTION OF THE SEVERAL VIEWS OF THE DRAWINGS

FIG. 1A is a schematic cross-sectional view of an aspherical lenticule according to one embodiment of the invention suitable for implantation in a patient's cornea,

FIG. 1B is an exaggerated graphical illustration of an aspherical profile of a posterior surface of the lenticule of FIG. 1A relative to a putative spherical profile,

FIG. 2 is a schematic cross-sectional view of a portion of the human eye including the cornea from which a flap is removed to expose a stromal surface on which the lenticule of FIG. 1A is placed during a keratophakia procedure,

FIG. 3 is a flow depicting various steps of a procedure according to the teachings of the invention for correcting a refractive error of a patient's eye,

FIG. 4A presents graphs depicting modulation transfer functions for an aspherical lenticule according to the teachings of the invention and a comparable spherical lenticule calculated in air at a wavelength of about 550 nm and pupil size of about 3 mm,

FIG. 4B presents graphs depicting modulation transfer functions corresponding to the aspherical and spherical lenticules for which similar data presented in FIG. 4A, calculated in air at a wavelength of about 550 nm and a larger pupil size of about 5 mm,

FIG. 5A presents graphs depicting modulation transfer functions for a model eye with an aspherical lenticule, a spherical lenticule and without a lenticule, each calculated at a wavelength of about 550 nm, and a pupil size of about 3 mm by assuming a perfect fit between the lenticule and the corneal flap,

FIG. 5B presents graphs depicting modulation transfer functions for a model eye with an aspherical lenticule, a spherical lenticule and without any lenticules, each calculated at a wavelength of about 550 nm, and a pupil size of about 5 mm by assuming a perfect fit between the lenticule and the corneal flap,

FIG. 6A presents graphs depicting modulation transfer functions for a model eye with an aspherical lenticule, a spherical lenticule and without a lenticule, each calculated at a wavelength of about 550 nm, and a pupil size of about 3 mm by assuming that the corneal flap retains its original shape with the growth of stromal tissue filling a gap between the corneal flap and the lenticules,

FIG. 6B presents graphs depicting modulation transfer functions for a model eye with an aspherical lenticule, a spherical lenticule and without a lenticule, each calculated at a wavelength of about 550 nm, and a pupil size of about 5 mm by assuming that the corneal flap retains its original shape with the growth of stromal tissue filling a gap between the corneal flap and the lenticules,

FIG. 7A depicts a histogram corresponding to RMS wavefront errors, expected as a result of manufacturing imperfections, calculated for an aspherical lenticule by employing a Monte Carlo analysis,

FIG. 7B depicts a histogram corresponding to RMS wavefront errors, expected as a result of manufacturing imperfections, calculated for a spherical lenticule by employing a Monte Carlo analysis, and

FIG. 8 is a cross-sectional view of a lenticule according to another embodiment of the invention having an aspherical posterior surface and a generally convex anterior surface that can be shaped by ablation to a desired shape so that the lenticule can provide correction of a refractive error when implanted in a patient's eye.

### DETAILED DESCRIPTION OF THE INVENTION

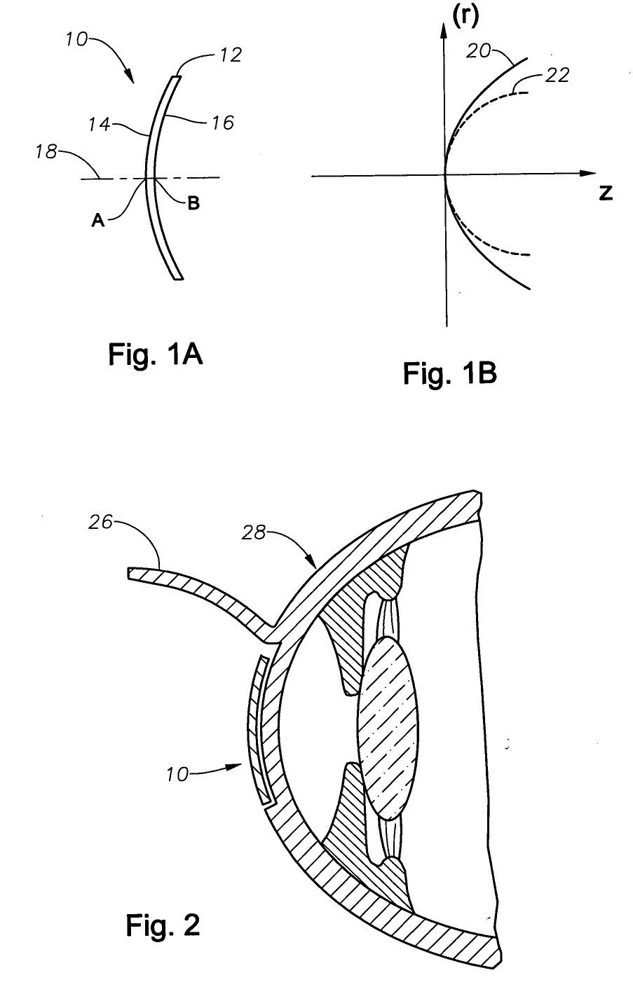

FIG. 1A schematically depicts a lenticule 10, herein also referred to as a corneal implant or a corneal inlay lens, for implantation in a patient's cornea that includes an optical body 12 defined by a anterior surface 14 and a posterior surface 16 adapted for placement on an internal corneal stromal surface, as discussed in more detail below. The lenticule 10 can be formed of a biocompatible optical material, i.e., a material that is compatible with corneal stromal tissue, that is photoablatable to allow modifying its shape, e.g., by employing laser light, such that it can provide a desired optical power while implanted in a patient's cornea. Some examples of suitable materials from which the lenticule can be formed include, without limitation, silicone, ploymethylmethacrylate, polyvinylpyrrolidine, optical homopolymers and copolymers or other suitable polymeric materials.

In this exemplary embodiment, the anterior surface 14, which is generally convex, and the posterior surface 16, which is generally concave, are symmetrical about an optical axis 18 that intersects the anterior and the posterior surfaces at points A and B, respectively. In other embodiments, one or both of the anterior and posterior surfaces can be asymmetric with respect to the optical axis. The exemplary lenticule 10 can have a central thickness, corresponding to the separation between points A and B, in a range of about 10 microns to about 300 microns and, more preferably, in a range of about 50 microns to about 100 microns. Further, the lenticule 10 can provide an optical power in a range of about −15 D to about +10 D, as measured in air. As discussed in more detail below, the lenticule 10 can acquire its shape upon photoablation of a portion thereof while placed against a corneal stromal bed exposed by removing a corneal flap. Alternatively, it can be shaped externally and then implanted in a patient's cornea.

With reference to FIG. 1B, the posterior surface 16 of the lenticule 10 is aspheric, characterized by an aspherical profile 20 (a profile of the surface as a function of radial distance (r) from the optical axis 18) that deviates from a putative spherical profile 22 that substantially coincides therewith at small radial distances (i.e., at locations close to the optical axis). In this exemplary embodiment, the posterior surface 16 exhibits a profile that is flatter than the putative spherical profile 22. In many embodiments, the asphericity of the posterior surface can be selected to substantially match an average asphericity depicted by the corneas of a selected group of individuals so as to improve the contact between the posterior surface of the corneal-implanted lenticule and an anterior surface of the corneal stromal bed, as described in more detail below. For example, the asphericity of the posterior surface can be different from the asphericity exhibited by the surface of the corneal stromal bed by less than about 50%, and more preferably by less than about 20%.

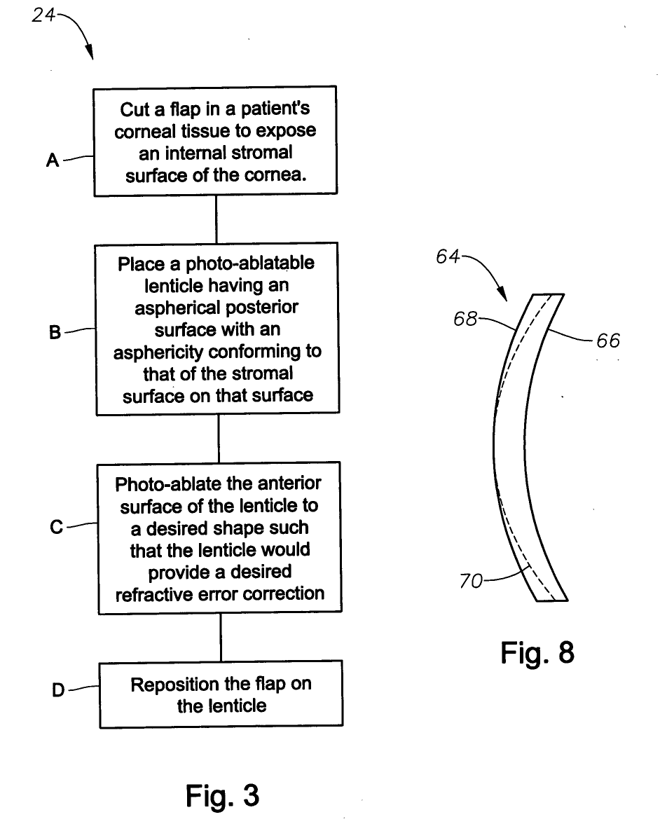

As noted above, the lenticule 10 can be implanted in a patient's cornea to function as a contact lens inlay. With reference to FIG. 2 and a flow chart 24 of FIG. 3, in one exemplary method for correcting a refractive index defect of a patient's eye, in an initial step A, a substantially uniform layer of tissue in the form of a flap 26 is cut, e.g., by micro-keratome, from a patient's cornea 28 to expose an anterior stromal surface 30 of the cornea. It is well known that the corneal anterior surface of the eyes of many individuals has an aspheric shape that can be characterized on average as a problate ellipse having a conic constant of about −0.25. Formation of a corneal flap with a substantially uniform thickness transfers the asphericity of the anterior corneal surface to the stromal bed.

The transfer of the asphericity of the corneal surface to the stromal bed can be also understood by considering the following mathematical formulation. The elliptical shape of the cornea can be described by the following relation:
r²=x²+y²=2r₀z−pz² Equation (1),
wherein x, y, and z are Cartesian coordinates corresponding to locations on the surface, r₀ is the apical radius, and p is the eccentricity of the ellipse. A comparison of the above Equation (1) with the more familiar following elliptical formula:

$$\frac{{x}^{2} + {y}^{2}}{{a}^{2}} + \frac{{( z - {z}_{0} )}^{2}}{{b}^{2}} = 1 , \qquad Equation ( 2 )$$

in which a and b correspond to the short and the long axes of the ellipse, respectively, shows that a and b can be provided as functions of r₀ and p by the following relations:

$$\begin{matrix}a = \frac{{r}_{0}}{\sqrt{p}} , & Equation ( 3 ) \\ b = \frac{{r}_{0}}{p} . & Equation ( 4 )\end{matrix}$$

For an average cornea, the apical radius p can be about 7.70 mm and the eccentricity p can be about 0.750, giving rise to values of 8.891 mm and 10.267 mm for the coefficients a and b, respectively.

The removal of a uniform layer of tissue from the cornea can be modeled as reducing the short and the long axes of the ellipse (coefficients a and b) by a fixed amount (dt) to produce a new eccentricity coefficient p′ given by the following relation:

$${p}^{′} = \frac{{( a - dt )}^{2}}{{( b - dt )}^{2}} . \qquad Equation ( 5 )$$

For a corneal flap having a thickness of about 200 microns, the above model provides an accentricity coefficient (p′) for the stromal bed having a value of 0.745, substantially equal to the eccentiricty of 0.750 of the anterior corneal surface. The stromal bed will, however, have a smaller apical radius than that of the cornea. For example, cutting a 200 micron thick flap in a cornea having a radius of 7.70 mm can result in a radius of 7.50 mm for the stromal bed.

As noted above, the aspherical profile of the posterior surface of the lenticule is selected so as to conform with the asphericity of the stromal bed, thereby providing a substantially even contact interface contact between the two surfaces. Such conformity of the lenticule's posterior surface with the surface of the stromal bed provides a number of advantages over conventional lenticules that have spherical posterior surfaces, and hence do not provide a good fit with the aspherical stromal surface. In particular, a mismatch between a conventional spherical lenticule and the stromal bed can lead to creation of uneven pressure between the lenticule and the stromal bed, which can in turn adversely affect the corneal physiology. In addition, a mismatch between a spherical lenticule and the aspheric stromal bed can cause the central portion of the lenticule to bulge out, thus increasing the spherical aberration of the eye and degrading visual performance. Moreover, such a mismatch can render the surgical outcome unpredictable. Another disadvantage of conventional spherical lenticules is that they typically have a large central thickness because of relatively steep edges of spherical surfaces. As the permeability of ion transportation depends inversely on the lenticule thickness, a large central thickness can reduce ion transportation. In contrast, an aspherical lenticule according to the teachings of the invention can provide not only a better fit to the stromal bed surface but it can also be made thinner than conventional spherical lenticules to enhance ion transportation. It can also improve optical and visual outcome after surgery.

Referring again to the flow chart 24 of FIG. 3, subsequent to the placement of the lenticule on the stromal bed, in step B, the lenticule's anterior surface 14 can be photo-ablated (step C), e.g., by employing excimer laser radiation, while retaining the lenticule in place by utilizing tools and methods known in the art. The ablation of the anterior surface of the lenticule can modify its shape, and hence the shape of the cornea upon completion of the implantation, so as to correct a refractive error of the eye. Some examples of such refractive errors include, without limitation, myopia, hyermetropia or hyeropia and astigmatism. The photo-ablation can be performed in a central region of the anterior surface, in a peripheral portion of the surface, or both based on the type of refractive error that needs correction. For additional disclosure of laser ablation techniques as applied to intracorneal implants, see U.S. Pat. Nos. 4,840,175; 5,722,971; 5,919,185; 6,436,092 and 6,702,807, herein incorporated by reference.

In some other embodiments, the photo-ablation of the lenticule can be performed external of the cornea to impart a desired shape thereto followed by its implantation in the cornea by formation of a flap in the corneal tissue.

After completion of the ablation process, in step C, the corneal flap is repositioned over the lenticule to lie over the lenticule's anterior surface in a relaxed state. Subsequent corneal healing results in retention of the lenticule within the corneal tissue. In some embodiments, not only the lenticule's posterior surface but also its anterior surface (e.g., surface 14 of the above exemplary lenticule 10), which is in contact with the inner surface of the flap, has an aspherical profile so as to substantially conform with the inner surface of the flap.

In some embodiments of the invention, the aspherical profile of the posterior surface of the exemplary lenticule 10 can be defined by the following relation:

$$z = \frac{{cr}^{2}}{1 + \sqrt{1 - ( 1 + k ) {c}^{2} {r}^{2}}} ,$$

wherein

z denotes a sag of the surface parallel to an axis (z) perpendicular to the surface,

c denotes a curvature at a vertex of the profile (e.g., at point B of the lenticule shown above in FIG. 1A),

k denotes a conic coefficient, and

r denotes a radial position on the surface.

In some embodiments, the conic constant k can be selected to be in a range of about −0.5 (corresponding to an asphericity exhibited by a cornea having extreme flattening) to about +0.2 (corresponding to an asphericity exhibited by a cornea having steepening). For example, the conic constant can be −0.25 (corresponding to asphericity often reported for an average cornea), although other conic constants can also be employed.

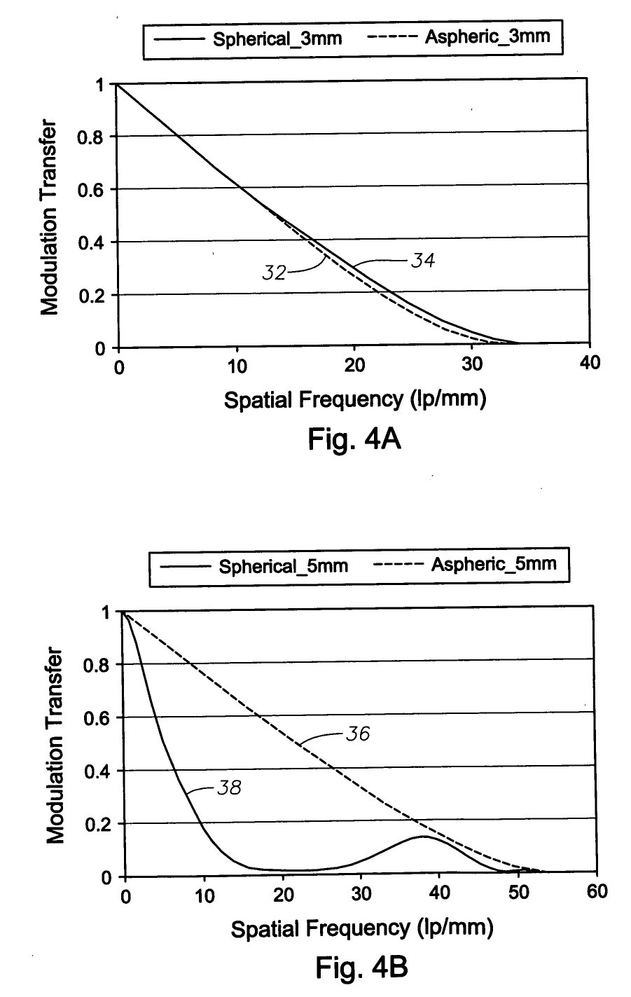

FIG. 4A present comparative data for modulation transfer functions in air calculated for an exemplary aspherical lenticule according to one embodiment of the invention having an optical power of about 6 D (graph 32) and a substantially identical lenticule that is spherical (graph 34), i.e., a lenticule having the same parameters as those of the aspherical lenticule but lacking its asphericity. The MTFs were calculated for a plurality of spatial frequencies and for a wavelength of about 550 nm and a pupil size of about 3 mm. FIG. 4B presents graphs 36 and 38 illustrating similar MTF data, respectively, for the aspherical lenticule and the corresponding spherical lenticule at a larger pupil size of about 5 mm. The lenticules were assumed to be formed of a hydrogel copolymer with a refractive index of 1.42, and to have an aspherical posterior surface with a radius of curvature of 7.50 mm at its vertex and an asphericity characterized by a conic constant of −0.25, and an aspherical anterior surface having a radius of curvature of 6.828 mm at its vertex and an asphericity characterized by a conic constant of −0.38. Further, the focal plane for each lenticule was chosen at a location corresponding to a minimum wavefront error.

The data presented in FIGS. 4A and 4B shows that the modulation transfer functions of the spherical and the aspherical lenticules are substantially similar for small pupil sizes (3 mm in this case). However, for a larger pupil size of 5 mm, the aspherical lenticule exhibits much higher MTF values, that is, its optical performance significantly exceeds that of the spherical lenticule. In fact, the aspheric lenticule exhibits nearly diffraction-limited optical properties while the aspheric lenticule shows very poor MTF performance because of a substantial spherical aberration. The superior performance of an aspherical lenticule according to the teachings of the invention at larger pupil sizes can be particularly advantageous as the ASK procedue is typically performed on a younger patient population than cataract patients, and hence large pupil sizes are expected.

In addition to the above calculated optical performance data in air, optical characteristics of these exemplary spherical and aspherical ASK lenticules were also simulated in a hypothetical model eye. The following two conditions corresponding to two extremes of conformity of the corneal flap with the lenticule were employed for the simulations: (a) the corneal flap was assumed to perfectly fit the lenticule, (b) the corneal flap was assumed to retain its original shape with the growth of stromal tissue filling a gap between the corneal flap and the lenticule. In a natural eye, the degree of conformity of the lenticule with the corneal flap falls between these two extreme conditions.

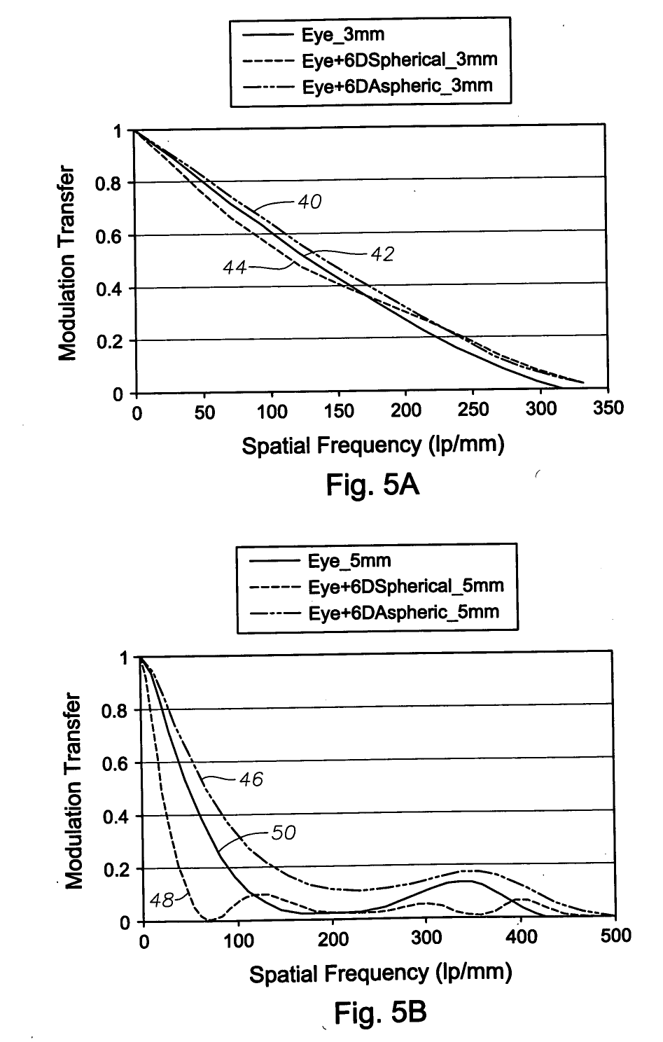

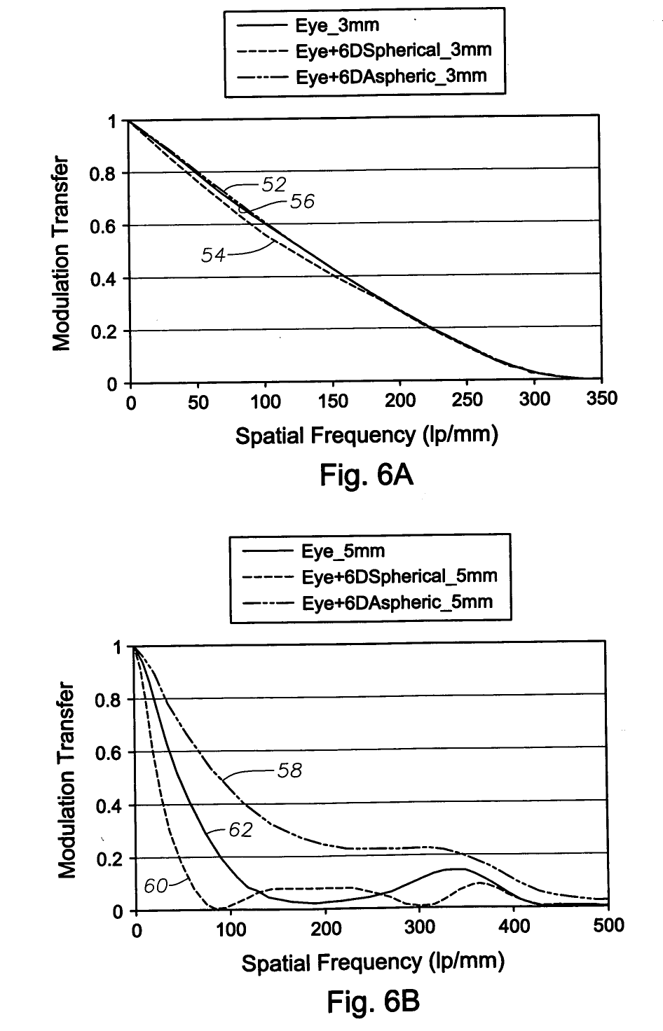

FIG. 5A presents graphs 40, 42, 44, where graph 40 exhibits optical performance of the aspherical lenticule under condition (a) in the model eye, as characterized by a modulation transfer function calculated at a plurality of spatial frequencies for a wavelength of about 550 nm and a pupil size of about 3 mm, while graph 44 exhibits corresponding MTF values for a substantially identical but spherical lenticule. Graph 42 depicts the optical performance of a model eye without an implant, presented as control data. FIG. 5B presents graphs corresponding to similar MTF data for a model eye with a aspherical lenticule (graph 46), a spherical lenticule (graph 48), and without any lenticules (graph 50), obtained at a larger pupil size of about 5 mm under condition (a). Further, FIG. 6A shows respective MTF data for a model eye having an aspherical lenticule, a spherical lenticule and without any lenticules (graphs 52, 54, and 56 respectively) under condition (b) for a wavelength of about 550 nm and a pupil size of about 3 mm, while FIG. 6B depicts similar MTF data for a model eye with an aspherical lenticule according to the teachings of the invention (graph 58), with a spherical lenticule (graph 60) and without any lenticules (graph 62) and a pupil size of about 5 mm.

The above data indicates that under both conditions (a) and (b), for small pupil sizes (3 mm in this example) there is no significant difference in optical performance between a model eye having an aspherical lenticule, a spherical lenticule or no lenticule at all. However, for a larger pupil size of 5 mm, the model eye with an aspherical lenticule provides a much superior optical performance relative to the model eye having a substantially identical spherical lenticule or no lenticule at all. In particular, under condition (a) and at a spatial frequency of about 100 lp/mm, which corresponds to rougly 30 cycles/degree, the model eye implanted with an aspherical lenticule exhibits a modulation transfer function that is about 2.16 times greater than that exhibited by the model eye having no lenticules and about 4.74 times greater than that exhibited by the model eye implanted with a substantially identical lenticule having a spherical shape. In other words, translating the improvement in the modulation transfer function to enhancement in contrast sensitivity, the eye with the aspheric lenticule has a 0.334 log unit contrast sensitivity gain over the eye with no lenticule, and 0.676 log unit gain over the eye implanted with the spherical lenticule.

Similar improvements are observed under condition (b) for a pupil size of about 5 mm. For example, the eye implanted with the aspheric lenticule exhibits a modulation transfer function at a spatial frequency of about 100 lp/mm that is 3.022 times greater than that exhibited by a model eye without a lenticule (a contrast sensitivity gain of about 0.48 log unit).

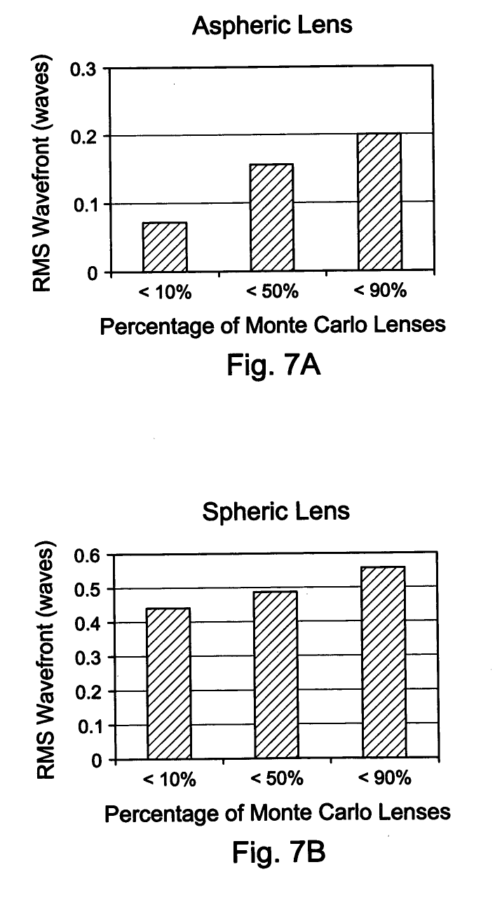

Although the optical performance of a manufactured spherical lenticule may be somewhat diminished relative to a theoretically expected performance due to manufacturing imperfections, under current assumed manufacturing tolerances, an aspherical lenticule is still expected to show significant comparative advantages over a conventional spherical lenticule. For example, Monte Carlo tolerance analysis that considers a number of factors (e.g., relative tilt between anterior and posterior surfaces and others) performed for aspherical and spherical lenticules (200 trials) show that aspherical lenticules can have an average root-mean-square (RMS) wavefront error of 0.146 waves with a standard deviation of 0.048 waves. The RMS wavefront error (or RMS error) is the root-mean-square wavefront deviation of a lenticule from a perfect plane wave. More particularly, 10% of the simulated aspherical lenticules showed an RMS error less than 0.072 waves, 50% showed an RMS error less than 0.159 waves and 90% showed an RMS error less than 0.200 waves, as shown schematically in FIG. 7A. In contrast, 10% of simulated spherical lenticules showed an RMS error less than 0.444 waves, 50% showed an RMS error less than 0.487 waves and 90% showed an RMS error less than 0.561 waves, as shown schematically in FIG. 7B. In fact, the optical quality of the best 10% of spherical lenticules (those exhibiting 0.430 to 0.444 waves RMS error) was worse than that of the worst 10% of the aspherical lenticules (those exhibiting 0.200 to 0.217 waves RMS error).

By way of another example, FIG. 8 schematically depicts a lenticule 64 according to another embodiment of the invention having an aspherical posterior surface 66 and a generally concave anterior surface 68. Similar to the previous embodiment, the lenticule 64 is formed of a photo-ablatable material that be shaped through ablation. For example, the anterior surface 68 can be ablated to modify the curvature of the anterior surface so as to generate a steeper convex surface 70 (shown by dashed lines) with or without asphericity.

An aspherical lenticule according to the teachings of the invention can be manufactured by employing techniques known in the art. For example, a top wafer and a bottom wafer of a suitable material, such as those recited above, can be pressed against one another, and subsequently shaped so as to generated a lenticule according to the teachings of the invention.

Those having skilled in the art will appreciate that various modifications can be made to the above embodiments without departing from the scope of the invention.

What is claimed is:

1. A lenticule for implantation in a subject's cornea for correcting a refractive error of the subject's eye, comprising:
a posterior surface adapted for placement on an internal stromal surface of the cornea, said posterior surface having an aspheric profile substantially matching an asphericity exhibited by the corneal stromal surface, and
an anterior surface opposed to said posterior surface, said anterior surface being photoablatable.

2. The lenticule of claim 1, wherein said lenticule exhibits a modulation transfer function in air greater than about 0.2 at a spatial frequency of about one-half a cut-off spatial frequency, a wavelength of about 550 nm and an aperture of about 5 mm.

3. The lenticule of claim 1, wherein a model eye in which said lenticule is implanted exhibits a modulation transfer function greater than about 0.2 at a spatial frequency of about 100 lp/mm, a wavelength of about 550 nm and a pupil size of about 5 mm.

4. The lenticule of claim 1, wherein said aspheric profile is characterized by the following relation:
$$z = \frac{{cr}^{2}}{1 + \sqrt{1 - ( 1 + k ) {c}^{2} {r}^{2}}} ,$$
wherein
z denotes a sag of the surface parallel to an axis (z) perpendicular to the surface,
c denotes a curvature at a vertex of the profile,
k denotes a conic coefficient, and
r denotes a radial position on the surface.

5. The lenticule of claim 4, wherein the the conic coefficient (k) is in a range about −0.5 to about +0.2.

6. The lenticule of claim 1, wherein said posterior surface has a generally concave shape.

7. The lenticule of claim 6, wherein said anterior surface has a generally convex shape.

8. The lenticule of claim 1, wherein said lenticule provide an optical power in air in a range of about −15 D to about +10 D.

9. An intracorneal implant, comprising:
an optic having a posterior surface and an anterior surface, said posterior surface being adapted for placement against an stromal surface of the cornea, said posterior surface having an aspherical profile substantially matching a contour of said stromal surface,
wherein said anterior surface is photo-ablatable so as to allow adjusting a refractive correction provided by said optic.

10. The implant of claim 9, wherein said optic is formed of a biocompatible polymeric material.

11. A lenticule for implantation in a patient's cornea, comprising
an optic having a posterior surface and an anterior surface, said posterior surface being adapted for placement on a generally convex internal corneal stromal surface having an asphericity substantially similar to that of the anterior corneal surface,
said posterior surface having an aspherical concave profile having an asphericity substantially similar to an average asphericity exhibited by the cornea of the eyes of a selected group of patients so as to facilitate positioning of said posterior surface against said stromal surface,
wherein said anterior surface of the lenticule is photoablatable to allow configuring an optical power of said optic for providing a desired refractive error correction.

13. The lenticule of claim 11, wherein said asphertical profile is characterized by a conic constant in a range of about −0.2 to about −0.5.

14. The lenticule of claim 11, wherein said optic provides an optical power in a range of about −15 Diopters to about +10 Diopters.

15. A method of correcting a refractive error of a subject's eye, comprising:
cutting a substantially uniform flap in the subject's corneal tissue to expose an internal stromal surface of said cornea,
providing a photoablatable lenticule having a posterior surface exhibiting an aspheric curvature substantially matching an asphericity exhibited by said exposed stromal surface,
placing said lenticule on said exposed stromal surface such that said aspheric surface of the lenticule is in contact with said exposed surface,
photoablating said lenticule to a selected shape for providing a desired refraction correction, and
repositioning said flap on the lenticule.

16. The method of claim 14, wherein said photoablating step comprises ablating a posterior surface of said lenticule opposed to said aspherical anterior surface.

17. The method of claim 15, wherein said photoablaing step comprises ablating a peripheral portion of said posterior surface.

18. A method of correcting a refractive error of a subject's eye, comprising
cutting a flap in the patient's corneal tissue to expose an internal stromal surface of the cornea,
providing a photoablatable lenticule including an anterior surface and a posterior surface, said posterior surface having an aspherical profile substantially matching an average convex aspherical profile of the corneas of a selected group of subjects,
placing said lenticule on the exposed stromal surface such that said aspherical posterior surface is in contact with the exposed corneal surface,
photoablating said anterior surface of the lenticule to a desired shape such that said lenticule provides a desired refractive correction, and
repositioning the flap on the lenticule.

19. The method of claim 17, further selecting said lenticule to have a central thickness in a range of about 100 microns to about 200 microns.
